SEQUENCE LISTING (22 sequences)

SEQ ID NO: 1 - DNA, 4557 bp, Oryza sativa
  promoter (1)..(2215)
  TATA_signal (2189)..(2195)
  CDS (2334)..(3695)
  Intron (3696)..(3888)
  CDS (3889)..(3996)
  polyA_signal (4512)..(4517)
catacctgttcaactgcagcgatattagaacatccagttccagccatcaccaatttaaccgatatatgatcatactttgatctgtctgaagatttcttcaggtcctttgcttttgtttgagcattattgcttgtgctagctacattggcatctgcctgtaatttcataacggataaaattaagattagtaacagagcaagttgagctacaacaggatctcactgtccttccagggacacaaaacaaccaacagtttacaatttggacagtgagaaaccttgataataactggcattgcaggaccttgacatggatcttccctcagtctgacactattgtagtgctcaccctgatgatacgatctatattggaatcaaaacaaaggaacatgagaatgtatgtccttttactgccagtattagcatattagtttaaaacagcagaacacacattaacttacaaatgaatcatactggtagcttcacgatcagaaaagtttcttatgtaccatcgtggtgagttaagctgcaaatcattcagtaatggtacaggcatcaatcaaatgttacagtacgttctcttctatagaagaaaggctataatatgaaaatgagtacgaaaaataacagagaaccatcaggggatatataagcagcacattgatctatatcacccatgtgtttcaaaatacagactaggcaaggctcaactgcctagcgtgttattcataggttgaagaaatcatactgcaaagcttgtattgaaaattaccatgtggatgcatatgttttttctcttgagaatagaagctgcttgcaactccatatgaccagcccaagtcccatcctcaagcatagagtcacaatatttctcaaacggttcctcgtcctcaatgaatggctcaaaatccacacggtgctcctgtaagatgcatacaagaaagattatgaagtgtaaataggccaatgcttgcttgcagcatgtcttttagataaaagaacaacccagcctggcagcctgtactctacgcccagatcagatgtacacagccaaagtttgcagcatgtgcaataccttattgattcaatggcaccctaattacagttcttaagtcacagtgttacaagatgtttcttggtcatagccaaagcttgcagtatttctaataccttattgattcaatggcaccctaactacagttattaagtcacagtgttacaagatgtttgttttggtcatctcatctgatgcataccttaatgtattgcacaatcattgcacggtacttcatgtgctcttcctcgttgccttcgagctggtcgcccattgccctgcagactacgatgagtaaccatataacacaagactgcataatgcatatactattcctcctttcataaactaacagtactctaacatatcgtacaacatcgattagcctcttcttgtaaaaagtggcaagaatttgagtaatggaatcgaaaagaagacctaaagaagcagttgccatccgcactaacttcgataatcttcaaccccagcgagtccagctgcgcccggaactccgtcatgtcggccttcttcgcgaacttcttctcctcctaacaaatcaatcaccaaaaggaaaaaaaaacgagaaaatatattagctaaagctcaattccccttccaccaaaaacgatccaatctccagctgactgaggcgcggggtattactgcatcacgcttcggcttccgggctttgggcgccgccacggctaccttcttcttgttccgagccattacgacacgtgcggtagtagtggagtctcgcctagatttccccgcggcggcggcggcggcgagggggaggggaggcggaatcgcagatagtatcaaatcgtactctaccagaagcccggagaagaaatcggatgggaaaaggaagaggagaagagaagagaagtcgtcaggtgatatttcgtgggccaaatgggccgggccgtaacacctcaatccccaatctgctacggcccgtgtgtgaacgtgacacgtcatcctatttagaatcgaataccgaacctgaacgtgacacgtcagatttaggagtagaaacgagtacactctacacgatacagatccaatacgagaccgacacgtcgtcagcgaccaagtaaaattcggtcacgaaccgtacgccaccaacctgtataaattcatcgaccgccaagcctctccagaacatagcacaagccaaccaaacaccgcacgatttcgtatccacacatacttctacgtgatttcgtttcgacgatctcgaggcgccgcggcgtgacgtgacgtcgacgacaaccatggcaggcggtggcggcaagtccgtagcggcggcgctggccatggcctgcttcctcctcatcctcgccgccttcgctcccccggcggcggcggcgccgccggacatcatgtcgatcatcaggtacaacgcggagcacggggtgcgggggctggagcggacggaggccgaggcgcgcgccgcgtacgacctgtggttggcgcggcaccggcgcggcggcggcggcggctcgcgcaacgggttcatcggcgagcacgagcgccggttccgcgtgttctgggacaacctcaagttcgtcgacgcccacaacgcccgcgccgacgagcgcggcgggttccgcctcgggatgaaccgcttcgccgacctcaccaacggcgagttccgcgccacctacctcggcaccacgcccgccggcagggggcgccgcgtcggggaggcgtaccgccacgacggcgtcgaggcgctgccggactccgtggactggagggacaagggcgccgtcgtcgcccccgtcaagaaccagggccagtgcggtagctgctgggcgttctcggcggtcgccgccgtggagggcatcaacaagatcgtcaccggcgagctggtgtcgctgtcggagcaggagctggtggagtgcgcgaggaacgggcagaacagcggctgcaacggtgggatcatggacgacgcgttcgccttcatcgcccggaacggcggcctcgacacggaggaggactacccgtacacggccatggacggcaagtgcaacctcgccaagaggagccgcaaggtggtgtccatcgacggcttcgaggacgtgcccgagaacgacgagctgtcgctccagaaggccgtggcgcaccagcccgtcagcgtcgccatcgacgccggcggccgcgagttccagctctacgactccggcgtgttcaccggccggtgcggcaccaacctggaccacggcgtggtggcggtggggtacggcacggacgccgccaccggcgccgcctactggacggtgcgcaactcgtgggggcccgactggggcgagaacggctacatccgcatggagcgcaacgtcaccgcgcgcaccggcaagtgcggcatcgccatgatggcgtcctacccgatcaagaaggggcccaacccgaagccgtcgccgccgtctccggcgccatcgccgccgcagcaatgcgaccggtacagcaagtgcccggcggggaccacctgctgctgcaactacgggatcaggaaccactgcatcgtgtggggatgctgccccgtcgagggcgccacctgctgcaaggatcactccacctgctgccccaaggagtatcccgtctgcaacgccaaggctcgcacttgctccaaggttttaaatttaaattttatgtttacttttaatttttagagttgattttagcgtttttataagtaatttctttttcatcattgacttttttttaagttgttaagaacatatgtaaaagttacattggccgctaatacgtgttatgtctgtgtgtgttatgtgctcacacgttgccatgttttttccttgacagagcaagaacagcccgtacaatatcaggactccggcggcgatggcacgaagtgttccggaacaacctgattcaatctcttttgtagttttgaatagggaagatctagtatagaagccttatctttgttactgttaccgagtcctttattattatcgctctttttttttcgcaagatgtataaagtcctaaacagttactgttactattactgaagttattatctatctttggatatgagttctcccaagtacagcatcacgtgttgttacagctctatcgtttgttttttagggtgtgtttagtttacgaaaaaaaaattggtatcacatcgaacgtttgatcgacgttgaaaggggttttcggatacgaatgaaaaaactaatttcataactcgcttggaaaccgcgagacgaatttattaagtctaattaatccgtcattagcacatgttggttactgtagcatttatgactaatcatggactaattaggctcaaaagattcgtctcacgatttccatgtaaactgtgcaattagttttttaatctatatttaacgcccgatgcatgtgtccaaagattcgatgtaatatttttagagaaaaaaattgggaactaaattattaataaacgttacagctgacaaatgggattgtagcttttgactgagt

SEQ ID NO: 2 - protein, 454 aa, Oryza sativa
MAGGGGKSVAAALAMACFLLILAAFAPPAAAAPPDIMSIIRYNAEHGVRGLERTEAEARAAYDLWLARHRRGGGGGSRNGFIGEHERRFRVFWDNLKFVDAHNARADERGGFRLGMNRFADLTNGEFRATYLGTTPAGRGRRVGEAYRHDGVEALPDSVDWRDKGAVVAPVKNQGQCGSCWAFSAVAAVEGINKIVTGELVSLSEQELVECARNGQNSGCNGGIMDDAFAFIARNGGLDTEEDYPYTAMDGKCNLAKRSRKVVSIDGFEDVPENDELSLQKAVAHQPVSVAIDAGGREFQLYDSGVFTGRCGTNLDHGVVAVGYGTDAATGAAYWTVRNSWGPDWGENGYIRMERNVTARTGKCGIAMMASYPIKKGPNPKPSPPSPAPSPPQQCDRYSKCPAGTTCCCNYGIRNHCIVWGCCPVEGATCCKDHSTCCPKEYPVCNAKARTCSK

SEQ ID NO: 3 - protein, 36 aa, Oryza sativa
SKNSPYNIRTPAAMARSVPEQPDSISFVVLNREDLV

SEQ ID NO: 4 - protein, 490 aa, Oryza sativa
MAGGGGKSVAAALAMACFLLILAAFAPPAAAAPPDIMSIIRYNAEHGVRGLERTEAEARAAYDLWLARHRRGGGGGSRNGFIGEHERRFRVFWDNLKFVDAHNARADERGGFRLGMNRFADLTNGEFRATYLGTTPAGRGRRVGEAYRHDGVEALPDSVDWRDKGAVVAPVKNQGQCGSCWAFSAVAAVEGINKIVTGELVSLSEQELVECARNGQNSGCNGGIMDDAFAFIARNGGLDTEEDYPYTAMDGKCNLAKRSRKVVSIDGFEDVPENDELSLQKAVAHQPVSVAIDAGGREFQLYDSGVFTGRCGTNLDHGVVAVGYGTDAATGAAYWTVRNSWGPDWGENGYIRMERNVTARTGKCGIAMMASYPIKKGPNPKPSPPSPAPSPPQQCDRYSKCPAGTTCCCNYGIRNHCIVWGCCPVEGATCCKDHSTCCPKEYPVCNAKARTCSKSKNSPYNIRTPAAMARSVPEQPDSISFVVLNREDLV

SEQ ID NO: 5 - DNA, 2215 bp, Oryza sativa
catacctgttcaactgcagcgatattagaacatccagttccagccatcaccaatttaaccgatatatgatcatactttgatctgtctgaagatttcttcaggtcctttgcttttgtttgagcattattgcttgtgctagctacattggcatctgcctgtaatttcataacggataaaattaagattagtaacagagcaagttgagctacaacaggatctcactgtccttccagggacacaaaacaaccaacagtttacaatttggacagtgagaaaccttgataataactggcattgcaggaccttgacatggatcttccctcagtctgacactattgtagtgctcaccctgatgatacgatctatattggaatcaaaacaaaggaacatgagaatgtatgtccttttactgccagtattagcatattagtttaaaacagcagaacacacattaacttacaaatgaatcatactggtagcttcacgatcagaaaagtttcttatgtaccatcgtggtgagttaagctgcaaatcattcagtaatggtacaggcatcaatcaaatgttacagtacgttctcttctatagaagaaaggctataatatgaaaatgagtacgaaaaataacagagaaccatcaggggatatataagcagcacattgatctatatcacccatgtgtttcaaaatacagactaggcaaggctcaactgcctagcgtgttattcataggttgaagaaatcatactgcaaagcttgtattgaaaattaccatgtggatgcatatgttttttctcttgagaatagaagctgcttgcaactccatatgaccagcccaagtcccatcctcaagcatagagtcacaatatttctcaaacggttcctcgtcctcaatgaatggctcaaaatccacacggtgctcctgtaagatgcatacaagaaagattatgaagtgtaaataggccaatgcttgcttgcagcatgtcttttagataaaagaacaacccagcctggcagcctgtactctacgcccagatcagatgtacacagccaaagtttgcagcatgtgcaataccttattgattcaatggcaccctaattacagttcttaagtcacagtgttacaagatgtttcttggtcatagccaaagcttgcagtatttctaataccttattgattcaatggcaccctaactacagttattaagtcacagtgttacaagatgtttgttttggtcatctcatctgatgcataccttaatgtattgcacaatcattgcacggtacttcatgtgctcttcctcgttgccttcgagctggtcgcccattgccctgcagactacgatgagtaaccatataacacaagactgcataatgcatatactattcctcctttcataaactaacagtactctaacatatcgtacaacatcgattagcctcttcttgtaaaaagtggcaagaatttgagtaatggaatcgaaaagaagacctaaagaagcagttgccatccgcactaacttcgataatcttcaaccccagcgagtccagctgcgcccggaactccgtcatgtcggccttcttcgcgaacttcttctcctcctaacaaatcaatcaccaaaaggaaaaaaaaacgagaaaatatattagctaaagctcaattccccttccaccaaaaacgatccaatctccagctgactgaggcgcggggtattactgcatcacgcttcggcttccgggctttgggcgccgccacggctaccttcttcttgttccgagccattacgacacgtgcggtagtagtggagtctcgcctagatttccccgcggcggcggcggcggcgagggggaggggaggcggaatcgcagatagtatcaaatcgtactctaccagaagcccggagaagaaatcggatgggaaaaggaagaggagaagagaagagaagtcgtcaggtgatatttcgtgggccaaatgggccgggccgtaacacctcaatccccaatctgctacggcccgtgtgtgaacgtgacacgtcatcctatttagaatcgaataccgaacctgaacgtgacacgtcagatttaggagtagaaacgagtacactctacacgatacagatccaatacgagaccgacacgtcgtcagcgaccaagtaaaattcggtcacgaaccgtacgccaccaacctgtataaattcatcgaccgccaagcctct

SEQ ID NO: 6 - protein, 471 aa, Oryzain Beta
MAARAAAAAFLLLLIVVGHRASDMSIISYNAEHGARGLEEGPTEAEARAAYDLWLAENGGGSPNALGGEHERRFLVFWDNLKFVDAHNARADEGGGFRLGMNRFADLTNEEFRATFLGAKVAERSRAAGERYRHDGVEELPESVDWREKGAVAPVKNQGQCGSCWAFSAVSTVESINQLVTGEMITLSEQELVECSTNGQNSGCNGGLMADAFDFIIKNGGIDTEDDYPYKAVDGKCDINRENAKVVSIDGFEDVPQNDEKSLQKAVAHQPVSVAIEAGGREFQLYHSGVFSGRCGTSLDHGVVAVGYGTDNGKDYWIVRNSWGPKWGESGYVRMERNINVTTGKCGIAMMASYPTKSGANPPKPSPTPPTPPTPPPPSAPDHVCDDNFSCPAGSTCCCAFGFRNLCLVWGCCPVEGATCCKDHASCCPPDYPVCNTRAGTCSASKNSPLSVKALKRTLAKLNTHELIDNM

SEQ ID NO: 7 - protein, 454 aa, Douglas fir
MGILLLFAVLALSAMAGSASRADFSIISYDSQDLIGDDAIMELYELWLAQHKKAYNGLDEKQKKFSVFKDNFLYIHQHNNQGNPSYKLGLNQFADLSHEEFKAAYLGTKLDAKKRLSRSPSPRYQYSVGEDLPESIDWREKGAVTAVKNQGSCGSCWAFSTVAAVEGINQIVTGNLTSLSEQELVDCDTSYNQGCNGGLMDYAFQFIISNGGLDSEDDYPYKANNGSCDAYRKNAHVVTIDDYEDVPENDEKSLKKAAANQPISVAIEASGRAFQFYESGVFTSNCGTQLDHGVTLVGYGSESGIDYWLVKNSWGNSWGEKGFIKLQRNLEGASTGMCGIAMEASYPVKKGANPPNPGPSPPSPVKPPTVCDNYYSCPESNTCCCMYDFGGYCYAWGCCPLNSATCCDDHYSCCPSDHPVCDLDAQTCLKSRKDPFGTKMLKRTPAKPYWSLSG

SEQ ID NO: 8 - protein, 374 aa, Tobacco
MAKTIITTLLFALFSSLSYAIDMSIIDYKNNHYARKWTLQSDEDQVKNRYEMWLAEHGRAYNALGEKEKRFEIFKDNLRFIEGHNNSGNRTYKVGLNQFADLTNEEYRTMYLGTKSDARRRFVKSKNPSQRYASRPNELMPHSVDWRKRGAVAPIKNQGSCGSCWAFSTVAAVEGINQIVTGEMITLSEQELVDCDRVQNSGCNGGLMDYAFEFIISNGGMDTEKHYPYRGVEGRCDPVRKNYKVVSIDGYEDVPRNERALQKAVAHQPVCVAIEASGRAFQLYSSGVFTGECGEEVDHGVVVVGYGSEDGVDYWIVRNSWGTKWGENGYVKMERNVKKSHLGKCGIMTEASYPTKDSAINKRNTSKEEKISSI

SEQ ID NO: 9 - protein, 328 aa, Rape
MSIYLRWSLEHGKSNSNSNGIINQQDERFNIFKDNLRFIDLHNENNKNATYKLGLTIFANLTNDEYRSLYLGARTEPVRRITKAKNVNMKYSAAVNVDEVPVTVDWRQKGAVNAIKDQGTCGSCWAFSTAAAVEGINKIVTGELVSLSEQELVDCDKSYNQGCNGGLMDYAFQFIMKNGGLNTEKDYPYHGTNGKCNSLLKNSRVVTIDGYEDVPSKDETALKRAVSYQPVSVAIDAGGRAFQHYQSGIFTGKCGTNMDHAVVAVGYGSENGVDYWIVRNSWGTRWGEDGYIRMERNVASKSGKCGIAIEASYPVKYSPNPVRGTSSV

SEQ ID NO: 10 - protein, 480 aa, Zea mays
MAASTTAAAAASVLLLLLSLARGGGLMSIVSYGERTDEEARRMYAEWMAAHGRTYNAVGAEERRYQVFRDNLRYIDAHNAAADAGVHSFRLGLNRFADLTNDEYPATYLGARTRPQRDRKLGARYHAADNEDLPESVDWRAKGAVAEVKDQGSCGTCWAFSTIAAVEGINQIVTGDLISLSEQELVDCDTSYNQGCNGGLMDYAFEFIINNGGIDTEKDYPYKGTDGRCDVNRKNAKVVTIDSYEDVPANDEKSLQKAVANQPVSVAIEAAGTAFQLYSSGIFTGSCGTRLDHGVTAVGYGTENGKDYWIVKNSWGSSWGESGYVRMERNIKASSGKCGIAVEPSYPLKEGANPPNPGPSPPSPTPAPAVCDNYYSCPDSTTCCCIYEYGKYCFAWGCCPLEGATCCDDHYSCCPHDYPICNVRQGTSLMGKDSPLSLSVKATKRTLAKRTGLSPATQLTALKSSALRKRSVALGRQQPG

SEQ ID NO: 11 - DNA, 23 bp, Artificial Sequence
  Reverse primer designated from the transcript sequence of GUS located in the T-DNA region.  Used in inverse PCR to determine the T-DNA flanking sequence.
ttggggtttctacaggacgtaac

SEQ ID NO: 12 - DNA, 24 bp, Artificial Sequence
  Forward primer designated from the transcript sequence of GUS located in the T-DNA region.  Used in inverse PCR to determine the T-DNA flanking sequence.
gaacccgctcgtctggctaagatc

SEQ ID NO: 13 - protein, 20 aa, Artificial Sequence
  Consensus sequence of ERFNIN motif which presents in all of the papain family cysteine proteases with the exception of cathepsin B
EXXXRXXXFXXNXXXIXXXN

SEQ ID NO: 14 - DNA, 20 bp, Artificial Sequence
  Forward primer used to detect rCysP1 transcript
aagtgcaacctcgccaagag

SEQ ID NO: 15 - DNA, 21 bp, Artificial Sequence
  Reverse primer used to detect rCysP1 transcript
ccggagtcctgatattgtacg

SEQ ID NO: 16 - DNA, 20 bp, Artificial Sequence
  Forward OsActin primer used as control
tccatcttggcatctctcag

SEQ ID NO: 17 - DNA, 20 bp, Artificial Sequence
  Reverse OsActin primer used as control
gtacccgcatcaggcatctg

SEQ ID NO: 18 - DNA, 22 bp, Artificial Sequence
  Forward primer developed from the sequence of Oryzain Beta
tgacatcaacagggaaaatgct

SEQ ID NO: 19 - DNA, 21 bp, Artificial Sequence
  Reverse primer developed from the sequence of Oryzain Beta
gtgttcagtttagcgagcgtg

SEQ ID NO: 20 - DNA, 25 bp, Artificial Sequence
  rCysP1 gene-specific forward primer
atcgaaaagaagacctaaagaagca

SEQ ID NO: 21 - DNA, 29 bp, Artificial Sequence
  rCysP1 gene-specific reverse primer
aacttgaggttgtccctacaggacgtaac

SEQ ID NO: 22 - DNA, 23 bp, Artificial Sequence
  T-DNA border-specific reverse primer
ttggggtttctacaggacgtaac

(19) United States
(12) Patent Application Publication
(10) Pub. No.: US 2005/0160498 A1
(43) Pub. Date: Jul. 21, 2005

(54) Gene encoding cysteine protease and its promoter which are expressed specifically in rice anther, a method for producing male sterile rice by suppressing expression of the gene

(76) Inventors: Yong-Yoon Chung, Seoul (KR); Sang-Hyun Lee, Gyeongju-si (KR); Gyn-Heung An, Pohang-si (KR)

Correspondence Address: BIRCH STEWART KOLASCH & BIRCH, PO BOX 747, FALLS CHURCH, VA, 22040-0747, US

(21) Appl. No.: 10/896,169

(22) Filed: Jul. 22, 2004

(30) Foreign Application Priority Data
Jan. 16, 2004 (KR) 10-2004-0003156

(51) Int. Cl. A01H 1/00; C12N 15/82; C07H 21/04; C12N 9/48; A01H 5/00

(52) U.S. Cl. 800/287; 536/23.2; 435/212; 800/320.2

(57) ABSTRACT

The present invention relates to a novel gene encoding cysteine protease expressed specifically in rice anther, an anther-specific promoter of the gene and a method for producing male sterile rice by suppressing expression of the gene. The rCysP1 gene of the present invention is a novel gene encoding cysteine protease that is expressed in rice anther and thus involved in pollen development. Therefore, suppression of the gene expression makes it possible to prepare male sterile rice available to control of seed production because the suppression results in pollen generation in rice. In addition, the above gene of the present invention is available to other Gramineae and provides an advantage to use antagonistic traits, which can be shown by over-expression.

### TECHNICAL FIELD

The present invention relates to a novel gene encoding cysteine protease (rCysP1) and its promoter which are expressed specifically in rice anther and a method for producing male sterile rice by suppressing expression of the gene. More specifically, the present invention relates to a method for inducing male sterile character into plant by suppressing expression of the gene encoding cysteine protease by isolating a rice anther-specific cysteine protease gene from a pool of T-DNA insertional rice, Oryza sativa L., using T-DNA gene-trap system and revealing the gene function, which is associated with the pollen development.

### BACKGROUND ART

Among cysteine proteases that have been reported till now, there was no gene that is expressed in anther or has a function available to male sterile. Technology for induction of male sterile into plants that have been developed till now was, also, to use selective death of anther tissue by induction of foreign toxic gene. Therefore, the present invention, technology for induction of male sterile into rice using an anther-specific gene of rice, itself involved in pollen development, is novel and invaluable.

Rice plant is a plant producing rice that is a main food in more than one-third of all over the world comprising the Korea and is one of economically important crops. All researchers of the world pay attention to a technology that produces F1 hybrid by induction of male sterile to increase production of the plant. F1 hybrid by induction of male sterile in rice plant can be produced by a cytoplasm male sterile (CMS) method, the method, however, is uneconomical and impractical in use. Also, a method for developing male sterile transgenic plant by genetic engineering can be applied to rice. However, the method uses foreign toxic genes (bacteria or plant genes) with a tapetum-specific promoter to induce selective death of anther organs for male sterile.

Anther is a male reproductive organ of plants with flowers and comprised of tapetum, endothecium, connective tissue, and vascular bundle tissue. Anther, also, functions as pollen generation. Developmental process of anther divides into two stages: the first stage in which tetrad microspore is formed by meiosis of microspore mother cell after shape of anther is formed; and the second stage in which differentiation of pollen and anther, tissue degeneration, dehiscence and pollen releasing are occurred. Only some of various genes involved in this developmental process are specifically expressed in anther.

Cysteine protease belongs to a family of enzymes that play importance role in intracellular protein degradation in widely distributed systems; animals, plants, and microorganisms. Amino acids from the degradation of proteins can be recycled for new protein synthesis. Cysteine proteases (CysP) in higher plant are extensively studied in seed because these proteolytic enzymes are recognized as the major enzymes for the germination processes (Shutov and Vaintraub, Phytochemistry 26, 1557-1566, 1987; Ryan and Walker-Simmons, Proteins and Nucleic acids. Vol 6, 321-350 1991; Ho et al., Plant Physiol. 122, 57-66, 2000). Nutrient supply for seed germination can be achieved by hydrolyzing starch and storage proteins preserved in the endosperm. CysPs, therefore, have been reported as the major enzymes responsible for hydrolysis of the major storage proteins, hordeins and glutelin in barley and rice, respectively (Rostog and Oaks, Plant Physiol. 81, 901-906, 1986; Kato Minamikawa, Eur. J. Biochem. 239, 310-316, 1996). CysPs also contribute to cells undergoing programmed cell death (PCD) (Solomon et al., Plant Cell 11, 431-443, 1999): In soybean cells, the enzyme production was found to increase and regulate during PDC.

Accordingly, for production of male sterile rice without the problems of the prior art, the present inventors have screened genes involved in male sterile trait using T-DNA insertional lines of rice since 2002. As the result, unlike common cysteine proteases that are expressed specifically in seed and involved in seed germination, a novel cysteine protease that is expressed specifically in anther and thus involved in pollen development was identified.

### DISCLOSURE OF INVENTION

It is, therefore, an object of the present invention is to provide a novel gene encoding cysteine protease that is expressed specifically in rice anther and related to pollen development.

Another object of the present invention is to provide a promoter of the gene encoding cysteine protease which is active specifically in rice anther.

Further object of the present invention is to provide a method for producing male sterile rice by suppressing expression of the gene.

To achieve the above object, the present invention provides a novel gene encoding cysteine protease, which is expressed specifically in rice anther and involved in pollen development, wherein the gene has the nucleotide sequence of the SEQ. ID NO. 1 and the amino acid sequence of the SEQ. ID NO. 4.

To achieve another object, the present invention provides a promoter of the gene encoding cysteine protease, which is active specifically in rice anther, wherein the promoter has the nucleotide sequence of the SEQ. ID NO. 5.

To achieve the further object, the present invention also provides a method for producing male sterile rice by suppressing expression of the above gene.

The gene according to the present invention, rCysP1 gene, is a novel gene encoding cysteine protease that is isolated from the T-DNA insertional line of rice, expressed specifically in anther of rice and related to pollen development, and represented with nucleotides sequences of the SEQ. ID NO. 1. Schematic diagram of the rCysP1 gene is shown in FIG. 1a and the nucleotide sequence of the rCysP1 is shown in the SEQ. ID NO. 1.

In FIG. 1a, closed black rectangles in the right represent exons and the gray box in the left indicates promoter region of rCysP1 gene that was also the product of inverse PCR. Insertion site of the T-DNA is indicated by open inverted triangle. The T-DNA contained gus and hygromycin resistance (hph) genes between right (BR) and left (BL) borders. Also, arrows indicate three primers (a, b and c) used for genotyping T2 progeny.

Meanwhile, in the SEQ. ID NO. 1 indicating the nucleotide sequence of the rCysP1 gene, intron comprising GT and AG sequence at the 5′ and the 3′ terminals, respectively, exist between No. 454 codon and No. 457 codon of the putative amino acid coding region (between asterisks). In 5′ noncoding sequence of the coding region, the putative TATA box sequence, TATAAAT, exists between No. −139 base and No. −145 base and in 3′ noncoding region, the putative polyadenylation signals, AATAAA, exists between No. 2179 base and No. 2184 base. Also, anther-specific promoter of the rCysP1 gene exists between 5′ noncoding sequence comprising TATA box sequence, No. −119 base, and No. −2333 base. Cys¹⁸⁰, His²⁹⁷ and Asn³³⁹ of the coding region are consistent with catalytic triad conserved among cysteine proteases of papain family. The consensus sequence of ERFNIN motif that presents in all of the papain family cysteine proteases with the exception of cathepsin B (Karrer et al., 1993) is also found between Glu⁻⁸⁴ and Asn⁻¹⁰³ of the amino acid sequence of rCysP1. However, the rCysP1 gene is found to use Val instead of Ile (Akasofu et al., Nucleic Acids. Res. 17, 6733, 1989). T-DNA was inserted into the site between −86 base and −87 base of 5′ noncoding sequence (FIG. 1b, the vertical arrow). The ORF of the above coding region encodes a total of 490 amino acids and represented with the amino acid sequence of the SEQ ID NO. 4.

Database searches with the flanking sequence show that the ORF was located on contig8664 (http://btn.genomics.org.cn:8080/rice/), OSJNBa0043A12 (http://www.ncbi.nlm.nih.gov/), and AK107506 (http://cdna01.dna.affrc.go.jp/cDNA/). Homology search with the deduced amino acid sequence reveals that among the cysteine protease found in plants, the highest homology was occurred to the sequence of rice oryzain β with 89% and 69% of identity in nucleotide and amino acid sequences, respectively. Therefore, the gene of the present invention belongs to a papain family of cysteine proteases.

Expression of the rCysP1 gene in the present invention is controlled temporally and spatially. Spatially, the rCysP1 gene is highly expressed in anther organ of rice flower, but weakly or hardly in root and flower without anthers and not altogether in leaf organ. Specifically, the rCysP1 gene is expressed in only tapetum and pollen among anther organ but not in vascular bundle or connective tissue. Furthermore, the gene according to the present invention shows very limited amounts of its transcript accumulation in germinating seed and rCysP1-tagged T-DNA insertional lines of rice show pollen degeneration in the anther organ, suggesting that the rCysP1 gene plays an important role in pollen development. The other aspect, temporally, expression level of the rCysP1 gene increases as flower becomes matured. Specifically, the gene is expressed the highest in the matured flower. The result suggests that the rCysP1 gene of the present invention belongs to the late-expressed gene of anther in developmental stage of flower.

A rice anther-specific promoter of the rCysP1 gene of the present invention has the nucleotide sequence of the SEQ. ID NO. 5. The promoter can be obtained by common cloning method from the rice genomic DNA. For example, common RCR reaction is performed using primers designed properly according to the sequence of the SEQ. ID NO. 5.

As a genotyping analysis for T2 progeny of rCysP1-tagged T-DNA insertional rice, the T-DNA is co-segregated to next generation. The homozygous rcysp1 mutants show severe delay of growth and development; the seed germinations are delayed for about 7 to 9 days and the root and shoot are grown normally except for showing a dwarfism which results in reduced plant height. Specifically, in case of panicle, the mutant plants contain several flowers remained unfertilized, leaving the flowers in green and flowering is delayed approximately 15 days. Therefore, reduced number of seed formation is obtained from the rcysp1 mutant. In addition, the rcysp1 mutant plants show abnormal pollen development. The first detectable sign of abnormality is observed at the uni-nucleated pollen stage just releasing from the microspore stage. At this stage, very limited number of pollen is observed in the anther and several of them are undergoing cell death. This pollen degeneration becomes much severe when the pollen entered the vacuolated pollen stage and thus, anthers from the rCysP1 mutants then contain completely empty locule at the mature stage.

As known in the above, the rCysP1 gene of the present invention is a novel gene encoding cysteine protease that is expressed in rice anther and thus involved in pollen development. Therefore, suppression of said gene expression makes it possible to prepare male sterile rice available to control seed production because the suppression results in pollen generation in rice. In addition, the rCysP1 gene of the present invention is available to other Gramineae such as wheat, maize, Indian millet or orchard grass and provides an advantage to use antagonistic traits, which can be shown by over-expression.

The other aspect, rice has several tillers per plant and can be propagated asexually by artificial isolation of the tiller following by transfer of it. Male sterile rice prepared by the method using the rCysP1 gene in the present invention can also be propagated asexually in this matter.

The present invention is explained in greater detail through the following experimental cases. However, it should be understood that the scope of the present invention is not limited hereto.

### BRIEF DESCRIPTION OF THE DRAWINGS

The above objects, features and advantages of the present invention will become more apparent from the following detailed description when taken in conjunction with the accompanying drawings, in which:

FIG. 1 shows schematic diagram and sequence of rCysP1 gene comprising its promoter. In here, FIG. 1a shows rCysP1 gene and structure and T-DNA insertion site and FIG. 1b shows nucleotide and deduced amino acid sequences of rCysP1;

FIG. 2 shows comparison of the deduced amino acid sequence of cysteine proteases found in plants. FIG. 2a shows alignment of cysteine proteases and FIG. 2b shows phylogenetic tree of representative cysteine proteases observed in plants;

FIGS. 3a, 3b, 3c and 3d show genomic DNA blot and RT-PCR analysis of rCysP1. FIG. 3b shows transcript accumulation of rCysP1 gene in the various organs (anther, antherless flower, leaf and root) and FIG. 3c shows the temporal expression in different developmental stages of flower. Also, FIG. 3d shows rCysP1 transcript accumulation in germinating seeds;

FIGS. 4a, 4b, 4c, 4d and 4e show GUS expression of rCysP1 tagged rice, especially anther-specific expression of rCysP1 promoter. In here, FIG. 4c shows possibility of male sterile character because production of pollen was degenerated by suppressing expression of the rCysP1 gene;

FIGS. 5a, 5b, 5c, 5d and 5e show genotyping of rCysP1 T2 progeny and phenotype of rCysP1 homozygous mutant;

FIG. 6a shows morphometric analysis of fully-grown plants of rCysP1 mutant and wild type. FIG. 6b shows fertilization (seed formation) ratio per panicle. At this time, FIG. 6c and FIG. 6d show tetrazolium staining of the anthers from rCysP1 mutants and from wild type. In the anther from rCysP1 mutants, stained pollens are not observed because production of pollen is degenerated; and

FIG. 7a-7j show cytological analysis of developing anther from wild type and FIG. 7k-7t show that of developing anther from rCysP1 mutant.

### BEST MODE FOR CARRYING OUT THE INVENTION

### EXAMPLE 1

### Cloning and Sequence Analysis of rCysP1 Gene

(1) First, T-DNA insertional lines of rice (Oryza sativa L.) in which GUS gene transcript was randomly inserted into the plant genome were generated as described in Jeon et al. (Jeon et al., 2000, T-DNA insertional mutagenesis for functional genomics in rice, Plant J. 22, 561-570) and were screened GUS expression in T2 progeny of the T-DNA insertional lines of rice as described in Gothandam et al. (Gothandam et al., 2003, Identification of anther-specific gene expression from T-DNA tagging rice, Mol. Cells 15, 102-109). A T-DNA tagged line that showed GUS expression specifically in the anther was selected and further characterized (FIG. 4).

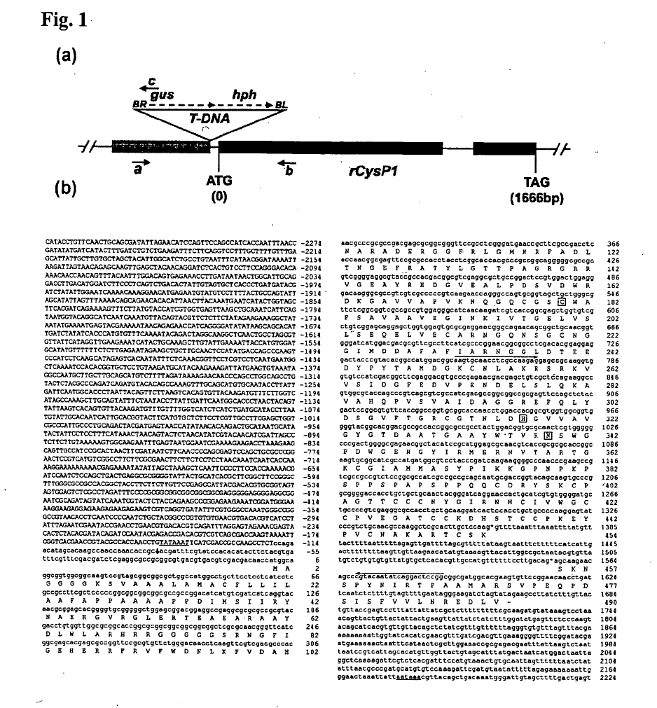

(2) Inverse PCR was performed to determine the T-DNA flanking sequence using a primer set designated from the transcript sequence of GUS located in the T-DNA region (FIG. 1a).

Inverse PCR was carried out as described in Triglia et al. (Triglia et al., 1988, A procedure for in vitro amplification of DNA segment that lie outside the boundaries of known sequences. Nucl. Acids Res. 16, 8186). One μg of genomic DNA was digested with Pst I restriction endonuclease for 12 h. The reaction was stopped by ethanol precipitation, and the DNA was resuspended in 40 μl of water. The resuspended genomic DNA was then self-ligated by T4 DNA ligase. PCR reaction was carried out in the 50 μl of mixture that contained 20 ng of DNA. 1×E×Taq buffer, 0.2 mM dNTP, 0.5 unit E×Taq polymerase (Takara, Japan), and 1 μM of primers. The primers used in the inverse PCR reaction were: 5′-TTG GGG TTT CTA CAG GAC GTA AC-3′(reverse) and 5′-GAACCCGCTCGTCTGGCTAAGATC-3′ (forward).

The inverse PCR result revealed that the T-DNA had been integrated into upstream region of a 1,666 bp-long open reading frame (ORF) in the rice genome. Database searches with the flanking sequence show that the ORF was located on contig8664 (http://btn.genomics.org.cn:8080/rice/), OSJNBa0043A12 (http://www.ncbi.nlm.nih.gov/), and AK107506 (http://cdna01.dna.affrc.go.jp/cDNA/).

(3) The genomic DNA was isolated and sequenced. And homology search with the amino acid sequences between the genomic DNA and cysteine proteases found in plants, Oryzain β (Oryza sativa, P25777), Zea mays (Zea mays, AAB70820.2), Douqlas fir (Pseudotsuga menziesii, JC4848), Tabacco (Nicotiana tabacum, TP3941) and Rape (Brassica napus, JQ1121) was carried out.

The result of sequence analysis is shown in FIG. 1 and the ORF encodes a total of 490 amino acids.

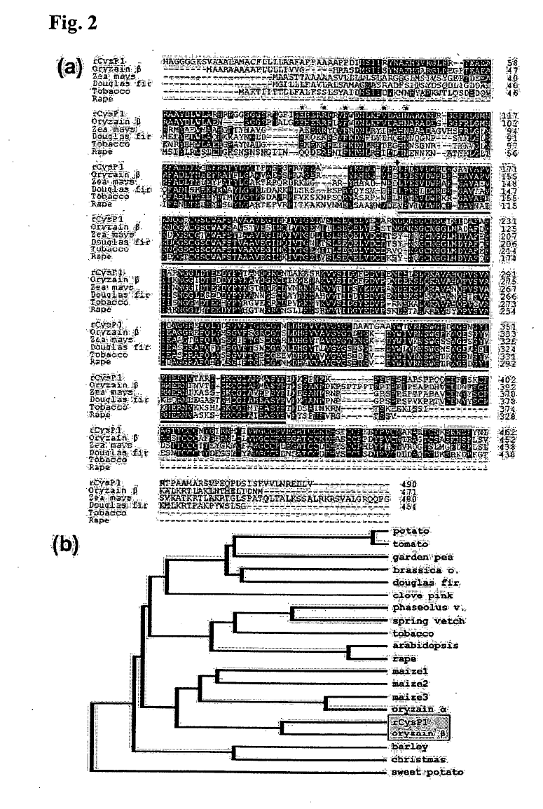

The result of homology search is shown in FIG. 2a. In FIG. 2a, identical amino acid residues are indicated by black-shaded box and similar ones are indicated by gray-shaded box. Asterisks indicate the consensus sequence of ERFNIN motif and peptidase C1 domain of papain family cysteine proteases is underlined. Putative posttranslational cleavage site is labeled with a vertical arrow.

The highest homology was occurred to the sequence of rice oryzain β with 89% and 69% of identity in nucleotide and amino acid sequences, respectively. Oryzain β, along with oryzain α and γ, belong to a papain family of cysteine proteases (Watanabe et al., J. of Biol. Chem. 266, 16897-16902, 1991). The rCysP1 gene contained the consensus sequence of ERFNIN motif which presents in all of the papain family cysteine proteases with the exception of cathepsin B (Karrer et al., Proc. Natl. Acad. Sci. USA 90, 3063-3067, 1993) and the consensus sequence was found between Glu⁻⁸⁴ and Asn⁻¹⁰³ of the amino acid sequence of rCysP1. As described in Karrer et al. (1993), it was found that the consensus sequence, EX₃RX₂ (V/I) FX₂NX₃IX₃N, was variable between species, although the two rice cysteine proteases, rCysP1 and Oryzain β, shared the same motif sequences (FIG. 2a). Unlike other cysteine proteases, however, two cysteine proteases were found to use Val instead of Ile as observed in mammalian system (FIG. 1b). Catalytic triad with Cys¹⁸⁰-His²⁹⁷-Asn³³⁹ conserved among members of papain family was observed in the amino acid sequence of rCysP1 (FIG. 1b). The result was consistent with the phylogenetic analysis among the representative cysteine proteases observed in plants. The phylogenetic analysis result is shown in FIG. 2b. The cysteine proteases used in the phylogenetic analysis were potato (Solanum tuberosum, CAB53515.1), tomato (Lycopersicon esculentum, YO6416), garden pea (Pisum sativum, S24602), brassica o. (Brassica oleracea, AAL60579.1), diuglas fir (Pseudotsuga menziesii, JC4848), clove pink (Dianthus caryophyllus, AAA79915.1), phaseolus v. (Phaseolus vulgaris, CAB17076.1), spring vetch (Vicia sativa, S47312), tobacco (Nicotiana tabacum, TO3941), arabidopsis (Arabidopsis thaliana, AAK92229.1), rape (Brassica napus, JQ1121), maizel (Zea mays, AAB70820.1), maize 2 (Zea mays, T01206), maize 3 (Zea mays, TO1207), oryzain α (Oryzain sativa, P25776), oryzain β (Oryzain sativa, P25777), barley (Hordeum vulgare, TO6208), Christmas (Sandersonia aurantiaca, AAD28477.1) and sweet potato (Ipomoea batatas, AAK27968.1).

Taken together, the results suggested that rCysP1 gene encodes a cysteine protease in rice.

(4) DNA bolt analysis was performed to determine genomic complexity of rCysP1 gene in the rice genome.

Rice leaves were pulverized in liquid nitrogen and suspended in an extraction buffer (100 mM Tris-HCl, pH 8.0, 50 mM EDTA, 500 mM NaCl and 1.25% SDS). After successive extraction with phenol/chloroform (1:1, v/v), the aqueous phase was concentrated by ethanol precipitation. The pellet was resuspended in TE buffer (10 mM Tris-HCl, pH 7.4, 1 mM EDTA). 13 μg of genomic DNA was digested with EcoR I, HindIII and Pst I enzyme and electrophoresed on a 0.8% agarose gel. The DNA was then blotted onto a nylon membrane after denaturation and neutralization. Baked membrane was pre-hybridized at 65° C. for 2 h, and hybridized with two ³²-P labeled rCysP1 gene specific probe for overnight. The two probes were generated from a full-length rCysP1 clone and a 5′-UTR region of the gene, respectively. After hybridization, the membrane was washed twice times with 2×SSC, 0.5% SDS for 5 min and twice times with 2×SSC, 0.1% SDS for 5 min at 65° C.

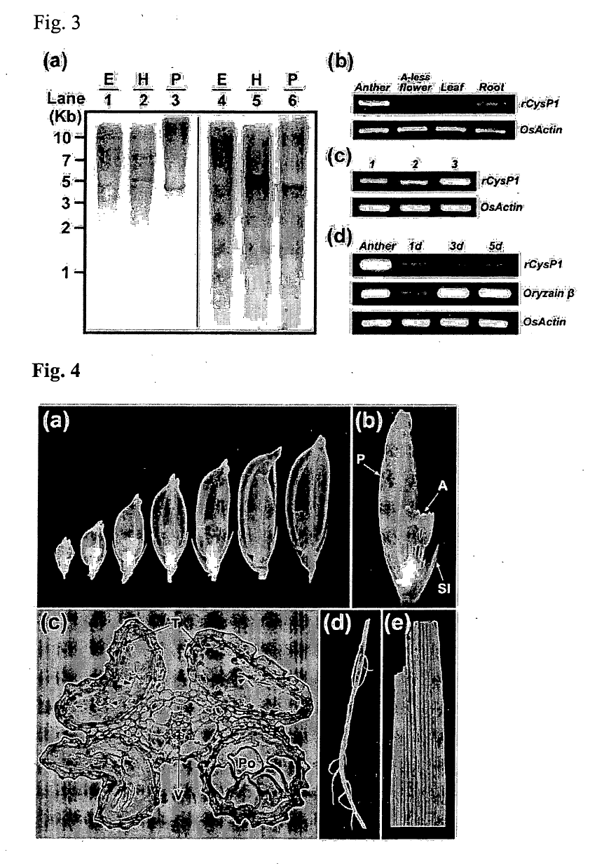

The result is shown in FIG. 3a. In the FIG. 3a, E represents EcoRI enzyme, H represents HindIII enzyme, and P represents Pst I enzyme. Lanes 1-3 show the result hybridized with the probe from the full-clone and lanes 4-6 show the result hybridized with the probe from the 5′-UTR region of the gene. Size markers are shown on the left.

The result showed that the probe derived from the full gene hybridized to more than one band, suggesting that rCysP1 gene exists as a small gene family in the rice genome (FIG. 3a). The probe from the 5′-UTR region of rCysP1 specifically recognized the rCysP1 gene in the genome (FIG. 3a right).

### EXAMPLE 2

### RT-PCR Analysis for Investigating Expression Pattern of rCysP1 Gene in Rice

RT-PCR analysis was performed to examine expression pattern of rCysP1 gene (FIG. 3).

(1) First, to investigate rCysP1 transcript accumulation in anther, anther-less flower, leaf and root of rice and temporal expression of the gene in rice flower at different developmental stages, total RNAs were extracted from the different organs of rice by the RNA isolation kit (TRI reagent, Molecular Research Center, Cincinnati, Ohio) and were used to synthesize cDNA templates together with reverse transcriptase. RT-PCR was performed with 94° C. incubation for 5 min followed by 30 cycles of 94° C. for 30 sec, 55° C. for 30 sec, and 72° C. for 50 sec, and 5 min extension at 72° C.

The primers to detect rCysP1 transcript were 5′-AAG TGC AAC CTC GCC AAG AG-3′ (forward) and 5′-CCG GAG TCC TGA TAT TGT ACG-3′ (reverse). OsActin primers used as control were 5′-TCC ATC TTG GCA TCT CTC AG-3′ (forward) and 5′-GTA CCC GCA TCA GGC ATC TG-3′ (reverse).

The result is shown in FIG. 3b and FIG. 3c. In FIG. 3c, lanes 1, 2 and 3 represent young flower, immature flower and mature flower, respectively. As shown in FIG. 3b, the gene transcript was highly accumulated in the anther, but very weakly in the root and leaf, and hardly in the flower without anthers. The result suggests that the gene is anther-preferential. Also, as shown in FIG. 3c, rCysP1 gene was expressed more in late developmental stage of rice flower.

(2) In addition, the rCysP1 gene expression during seed germination was compared with Oryzain β expression, since the rCysP1 showed homology with Oryzain β which was known to be active in germinating seed. Primers developed from the sequence of Oryzain β was used as a positive control to show that the PCR reaction was properly done.

| | |
|---|---|
| 5′ -TGACATCAACAGGGAAAATGCT-3′ | (forward) |
| 5′ -GTGTTCAGTTTAGCGAGCGTG-3′ | (reverse) |

The RT-PCR result is shown in FIG. 3d. In FIG. 3d, 1d, 3d, and 5d indicate one day, three days, and five days of germination, respectively. As shown in FIG. 3d, transcript of Oryzain β were highly accumulated during seed germination as previously studied (Watanabe et al., 1991). rCysP1 transcript accumulation was, however, rather preferential to the anther, showing very limited amounts of its transcript accumulation in germinating seed. The results suggested that germination process might not be a primary role of rCysP1 and the gene is involved in pollen development.

### EXAMPLE 3

### Determination of Activity of rCysP1 Promoter in Rice

Promoter activity of rCysP1 gene was determined by histochemical GUS assay using T2 generation of the rCysP1-tagged T-DNA rice (FIG. 4).

Various organs (flower, root and leaf) of rice were collected to perform GUS assay. The rice flower was also subdivided into two parts, anther and rest of the flower without anthers by eliminating the organ. Histochemical analysis of GUS activity in the present invention was performed as described by Jefferson et al. (Jefferson et al., 1987, GUS fusion: β-D-glucuronidase as s sensitive and versatile gene fusion marker in higher plants, EMBO J. 6, 3901-3907) with minor modifications. Samples were incubated overnight at 37° C. in a solution containing 100 mM sodium phosphate, pH 7.0, 0.1% 5-bromo-4-chloro-3-indolyl-β-D-glucuronide, 5 mM potassium ferrocyanide, 5 mM potassium ferricyanide, 10 mM EDTA, 0.5% Triton x-100, and 20% ethanol. Samples were then washed with 70% ethanol and examined under a stereomicroscope.

Also, to investigate temporally GUS expression in the anther, three flowers at different developmental stages (young flower, immature flower, and mature flower) were collected and GUS assay was performed. For further analysis of the promoter activity, the anther organ from the rCysP1 mutant were fixed in a solution containing 4% (w/v) paraformaldehyde, 0.5% (w/v) glutaraldehyde, and 100 mM phosphate buffer(ph 7.0) for overnight at 4° C. The samples were then dehydrated in an ethanol series and embedded in an acrylic resin (London Resin Company, London, UK). The resin-embedded flower samples were sliced into 1 μm sections with an ultra-microtome (LKB, Bromma 2088) and counter-stained with Safranin O. The tissue sections were examined under a light microscope (Zeiss).

The result is shown FIG. 4. FIG. 4a shows GUS expression in flowers at different developmental stages and FIG. 4b is to examine dissection of the anther with GUS expression under a light microscope and shows localization of rCysP1 gene. FIG. 4c also shows GUS expression (arrowheads) restricted in the anther locule. FIG. 4d and FIG. 4e show leaf and root that did not show GUS expression, respectively. In FIG. 4, A indicates an anther of rice and S1 indicates a sterile lemma. P indicates a palea and Po indicates pollen. T represents a tapetum and V represent a vascular bundle.

As shown in FIG. 4a, GUS expression was not visible in the immature flower. The highest level of the expression was obtained from mature flower. The result suggests that the gene belongs to the late-expressed gene of anther. Furthermore, as shown in FIG. 4b-4e, rCysP1 promoter was highly active in the anther but not in other organs of flower (FIG. 4b). GUS expression didn't be detected in the vascular bundle or the connective tissues. In the locules, GUS expression was observed in tapetum and some in developing pollen. However, contrary to the result in the example 2, GUS expression in other vegetative organs of rice, leaf and root, did not be detected (FIG. 4d and 4e). GUS expression was not also found in germinating seeds. Probably, this is because activity of rCysP1 promoter was too low to deliver a visual detection of GUS expression, although those organs (except for leaf) exhibited small amounts of the transcript accumulation (FIG. 3). The present inventors also found that anthers from the rCysP1-tagged T-DNA rice showed significant defect in pollen development, i.e. pollen degeneration (FIG. 4d). Taken together, the results suggested that rCysP1 gene is involved in pollen development.

### EXAMPLE 4

### Co-Segregation of T-DNA and Phenotypes of Homozygous Mutant

(1) In order to study genetic segregation of T-DNA, T2 progeny of the rCysP1-tagged T-DNA rice was generated and genotyped. Genomic DNAs extracted from young leaves of a total of 19 T2 progeny were used for genotyping to determine their homo- or heterozygosities. PCR reaction was performed by 35 cycles of 94° C. for 1 min, 57° C. for 1 min, and 72° C. for 2 min. The following primers a, b and c were used for the PCR reaction.

Primer a (rCysP1 gene-specific forward primer):

5′ -ATCGAAAAGAAGACCTAAAGAAGCA-3′

Primer b (rCysP1 gene-specific reverse primer):

5′ -AACTTGAGGTTGTCCCTACAGGACGTAAC-3′

Primer c (T-DNA border-specific reverse primer):

5′ -TTGGGGTTTCTACAGGACGTAAC-3′

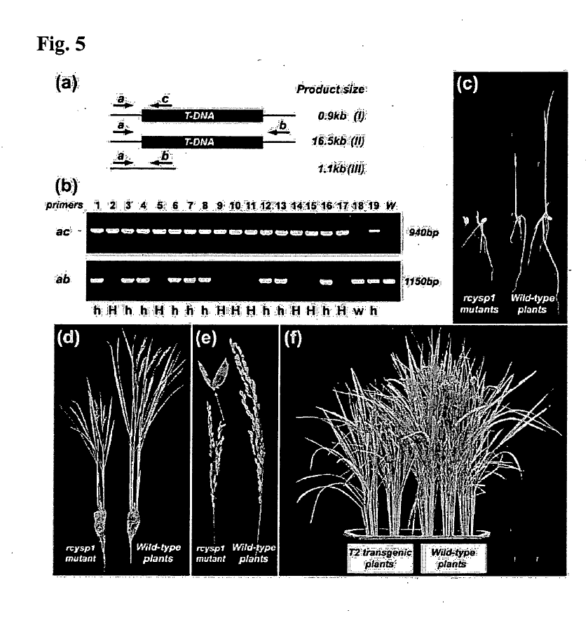

As indicated in a schematic diagrams of FIG. 5a, the primer a is a forward primer generated from upstream region of rCysP1 gene and the primer b is a reverse primer generated from coding region of rCysP1 gene. The primer c is, also, a reverse primer from T-DNA region. Combination of the primers a and c produces a 0.9 kb PCR fragment and the T2 plants who allow this amplification only would be homozygous because it is too large to amplify DNA fragment (16.5 kb) by the combination of the primers a and b (FIG. 5a). Wild-type plant, however, allows to produce single 1.1 kb PCR fragment by the primers a and b combination since there is no T-DNA insertion in the genome, while heterozygous T2 plants allow both 0.9 kb and 1.1 kb PCR amplifications by ac and ab combinations, respectively.

The result is shown in FIG. 5b. Lanes 2, 5, 9, 10, 11, 14, 15 and 17 were homozygous and lanes 1, 3, 4, 6, 7, 8, 12, 13, 16 and 19 were heterozygous. The plant 18 was wild type. These results demonstrated that the T-DNA has been co-segregated to next generation.

(2) The homozygous plants were further characterized in regards of growth and development because those plants were expected to deliver mutant phenotypes resulted from functional trapping of rCysP1 gene by T-DNA integration.

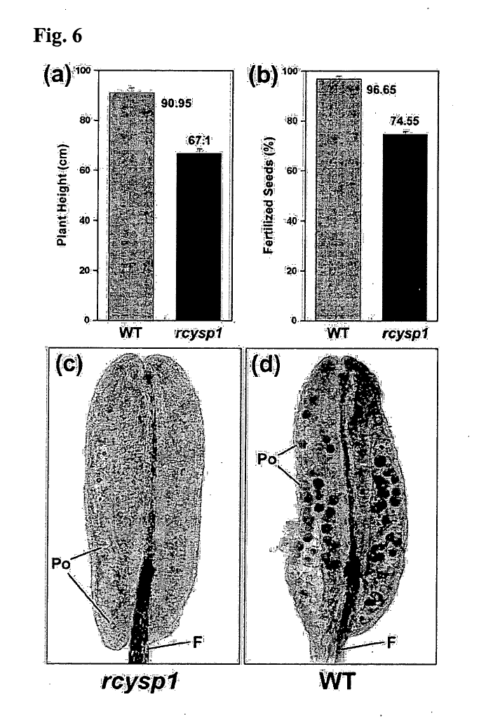

The result is shown in FIG. 5 and FIG. 6. In the homozygous rcysp1 mutants, the seed germinations were delayed for about 7 to 9 days but they eventually grew to fully mature plants. As the result of analyzing 17 fully grown rcysp1 mutants and 24 wild-type plants for their height, the mature rcysp1 mutant showed a dwarfism that resulted in reduced plant height (FIG. 5d, 5f and 6a). Normal root and shoot growth were observed in the mutant plants (FIG. 5c) but overall growth was severely retarded (FIG. 5f). Also, they formed normal panicles with flowers. The panicle, however, contained several flowers remained unfertilized, leaving the flowers stayed in green (FIG. 5e, arrow). Fertilization ratio per panicle in the rcysp1 mutant plants was lower than that in the wild type plants (FIG. 6b) and flowering was delayed approximately 15 days. This is probably due to the abnormal development of the anther.

The other aspect, many of the anthers from those homozygous plants were observed from tetrazolium staining under a light microscope. Tetrazolium staining was done by using a solution containing a 1% (w/v) aqueous solution of 2, 3, 5-triphenyltetrazolium chloride in 50% sucrose at 28° C. in darkness for 1 h. The result revealed that those homozygous plants did not contain viable pollen (FIG. 6c). Therefore, reduced number of seed formation was obtained from the rcysp1 mutant (FIG. 6b).

### EXAMPLE 5

### Cytological Features of rcysp1 Mutant During Pollen Development

Anther sections were further examined under a light microscope to investigate distinction between wild type and the rcysp1 mutant in regards of pollen development. To do this, anthers were divided into ten developmental stages, more specifically, two in pollen mother cell stage, two in tetrad stage, one in microspore stage, two in uni-nucleated pollen stage, one in vacuolated pollen stage, and two in mature pollen stage, and carefully investigated their cytological features.

Rice flowers from wild type and rcysp1 mutant were fixed in a solution containing 4% (w/v) paraformaldehyde, 0.5% (v/v) glutaraldehyde, and 100 mM phosphate buffer (pH 7.0) for overnight at 4° C. The samples were then dehydrated in an ethanol series and embedded in an acrylic resin (London Resin Company, London, UK). The resin-embedded flower samples were sliced into 1 μm sections with an ultra-microtome (LKB, Bromma 2088) and stained with 0.5% toluidine blue containing 0.1% sodium carbonate. The tissue sections were examined under a light microscope (Zeiss).

For investigation of GUS expression in the rCysP1-tagged anthers, anther samples were prepared and dissected as prescribed above and examined under a light microscope after staining with Safranin O. The anther samples were, also, observed with tetrazolium staining as described in example 4.

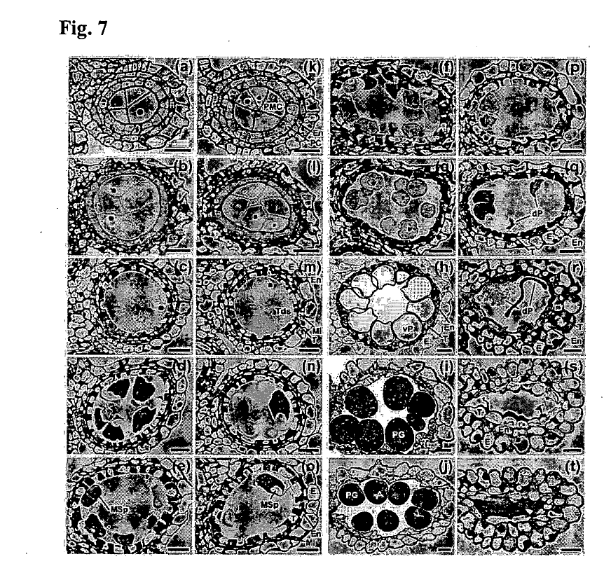

The results are shown in FIG. 7. In FIGS. 7, 7a, 7b, 7k, and 7l show the cytological features of anthers in the pollen mother cell stage. 7c, 7d, 7m, and 7n show those of anthers in the tetrad stage. 7e and 7o show those of anthers in the microspore stage. 7f, 7g, 7p, and 7q show those of anthers in the uni-nucleated pollen stage. 7h and 7r show those of anthers in the vacuolated pollen stage. Finally, 7i, 7j, 7s, and 7t show those of anthers in the mature pollen stage. Also, 7a-7j shows those of developing anthers from the wild type plants and 7k-7t show those of developing anthers from the rcysp1 mutants. dP indicates degenerated pollen and E indicates epidermis. En indicates endothecium and Ml indicates middle layer. MSp indicates microspores and PC indicates parietal cell. PG indicates pollen grains, PMC indicates pollen mother cell, and T indicates tapetum. Tds indicates tetrads and vMS indicates vacuolated pollen. The arrowheads indicate abnormal fibrous materials in the locule. Scale bars is 20 μm.

As observed in the above example 4, anthers from the rcysp1 mutant were found to contain abnormal pollen development (FIG. 7q-t). The first detectable sign of abnormality was observed at the uni-nucleated pollen stage just releasing from the microspore stage (FIG. 7q). At this stage, it was found that very limited number of pollen in the anther and several of them were undergoing cell death. This pollen degeneration became much severe when the pollen entered the vacuolated pollen stage where they lost their cytoplasm (FIG. 7r). Anthers from rcysp1 mutant then contained completely empty locule at the mature stage, whereas wild type anther contained fully mature pollen grains in the locule (FIG. 7i).

### Industrial Applicability

As described in the above, the rCysP1 gene of the present invention is a novel gene encoding cysteine protease that is expressed in rice anther and thus involved in pollen development. Therefore, suppression of the gene expression makes it possible to prepare male sterile rice available to control of seed production because the suppression results in pollen generation in rice. In addition, the above gene of the present invention is available to other Gramineae and provides an advantage to use antagonistic traits, which can be shown by over-expression.

1. A nucleotide sequence of gene encoding cysteine protease in rice having the SEQ. ID NO. 1, which is specifically expressed in anther of rice (Oryza sativa L.) and involved in pollen development.

2. An amino acid sequence of cysteine protease in rice having the SEQ. ID NO. 4, which is specifically expressed in anther of rice (Oryza sativa L.) and involved in pollen development.

3. A nucleotide sequence of a promoter of the gene encoding cysteine protease having the SEQ. ID NO. 5, wherein the promoter is specifically active in anther of rice (Oryza sativa L.).

4. A method for producing male sterile rice comprising the steps of:
(a) Suppressing expression of the gene encoding cysteine protease having the SEQ. ID NO. 1 by T-DNA insertion into rice genome;
(b) Selecting homozygous mutant plants showing dwarfism in plant height, delayed flowering and seed germination, and pollen degeneration among the T2 progeny; and
(c) Propagating asexually the selected mutant plants by their tiller.
